(19) United States
(12) Patent Application Publication
(10) Pub. No.: US 2013/0143323 A1
(43) Pub. Date: Jun. 6, 2013

(54) APPARATUS AND METHOD FOR DECELLULARIZING, RECELLULARIZING OR TREATING ORGANS

(71) Applicants: Geoffrey S. FROST, Toronto (CA); Thomas K. WADDELL, Toronto (CA)

(72) Inventors: Geoffrey S. FROST, Toronto (CA); Thomas K. WADDELL, Toronto (CA)

(21) Appl. No.: 13/486,529

(22) Filed: Jun. 1, 2012

Related U.S. Application Data
(60) Provisional application No. 61/492,161, filed on Jun. 1, 2011.

(51) Int. Cl. C12N 5/00

(52) U.S. Cl. CPC C12N 5/0062
USPC 435/378; 435/283.1

(57) ABSTRACT

A biomedical device and method provide for decullularization, recellularization or other treatment of an organ of a human or animal. To keep pressures to a minimum and to ensure that the perfusion fluid uniformly perfused the organ, the organ is supported and rotated during the perfusion process. The organ is supported by a medium, which may comprise a liquid or pallets in a vessel, with a vessel mounted for limited rotation. The perfusion tubing, for supply of perfusion fluid to inform the organ can be mounted both to a support structure and the vessel. The perfusion system can include tubing for supply of air or an air substitute.

### FIELD

This invention relates to an apparatus for and a method of decellularizing, recellularizing or treating lungs or other organs, from a human or animal body.

### BACKGROUND

The following paragraphs are not an admission that anything discussed in them is prior art or part of the knowledge of persons skilled in the art.

Recent work in the field of tissue engineering has led researchers to believe that it is possible to regenerate organs for use in transplantation. Achieving the goal of organ regeneration would greatly expand the donor pool for transplantation—at present a significant percentage of organs donated for transplantation are rejected due to concerns regarding the organ's suitability.

Current state of the art involves harvesting an unsuitable organ. The unsuitable organ is then treated with a detergent solution—the compositions of which have been illuminated in a variety of scientific papers. The detergent removes the cells from the organ, but leaves behind the extra-cellular matrix (ECM). The ECM is made of proteins and glycosaminoglycans. This remnant structure is then used as a building block for regenerating a new organ. The matrix is infused with cells, which are then encouraged to grow around the matrix and re-form the lost organ. By starting with specific cell types that encourage growth, an entirely new organ—free of past defects—can be created. This work has been carried out successfully in small animal models for a variety of organs.

### SUMMARY

The invention provides a method to deliver perfusion fluid to an organ undergoing decellularization. A device or apparatus that makes use of this method is also presented. The device intends to achieve complete perfusion in human-scale organs using a combination of hydrostatic pressure and a pre-determined input pressure. The method in question rotates the organ undergoing decellularization such that all sections of the organ are exposed to a maximum pressure. The maximum pressure can be varied by the user and should be low enough to prevent damage to the extracellular matrix of the organ in question, while high enough to achieve adequate perfusion.

The apparatus and method pertain to the fields of tissue engineering, pulmonary physiology, lung transplant, and organ generation and regeneration.

The apparatus and method are specifically interested in the decellularization of lungs. In the pulmonary case, lung decellularization and recellularization has been carried out in mouse and rat lungs. Attempts to scale up the concept of decellularization to larger human-scale models have not been published in scientific literature.

Decellularization of a large organ is not a trivial replication of the small animal case. In an aspect of this specification, a device that allows for complete decellularization of a human-scale lung is presented.

In accordance with a first aspect of the present invention, there is provided: a biomedical device for use in decellularizing, recellularizing or otherwise treating human or animal organs, the device comprising:

(a) a vessel to hold the organ being decellularized;

(b) a support structure that holds the vessel during rotation; and

(c) a mechanical component that rotates the vessel.

In accordance with a second aspect of the present invention, there is provided a method of supplying perfusion liquid to an organ, the method comprising:

(a) providing for a supply of a perfusion liquid to an organ;

(b) providing for at least limited rotation of the organ; and

(c) while rotating the organ, supplying perfusion liquid to the organ, to cause uniform perfusion of the liquid in the organ.

Other aspects and features of the teachings disclosed herein will become apparent, to those ordinarily skilled in the art, upon review of the following description of the specific examples of the specification.

### DRAWINGS

The drawings included herewith are for illustrating various examples of articles, methods, and apparatuses of the present specification and are not intended to limit the scope of what is taught in any way. In the drawings:

FIG. 1 is a perspective view of an apparatus for decellularizing lungs;

FIGS. 2A, 2B, 2C, and 2D are perspective, top, side, and front views, respectively, of a vessel used to house the lungs during decellularization;

FIGS. 3A, 3B, 3C, and 3D are side, front, top, and perspective views, respectively, of a central axis splitter;

FIGS. 4A, 4B, 4C, and 4D are perspective, top, front, and side views, respectively, of a support that holds the central axis splitter and vessel above a surface;

FIGS. 5A and 5B are side and front views, respectively, of the vessel showing the tubing entering the vessel;

FIG. 6 is a side of the apparatus; and

FIG. 7 is a side view of an alternative embodiment of the apparatus

### DETAILED DESCRIPTION

It will be appreciated that for simplicity and clarity of illustration, where considered appropriate, reference numerals may be repeated among the figures to indicate corresponding or analogous elements. In addition, numerous specific details are set forth in order to provide a thorough understanding of the examples embodiments described herein. However, it will be understood by those of ordinary skill in the art that the example embodiments described herein may be practiced without these specific details. In other instances, well-known methods, procedures and components have not been described in detail so as not to obscure the example embodiments described herein. Also, the description is not to be considered as limited to the scope of the example embodiments described herein.

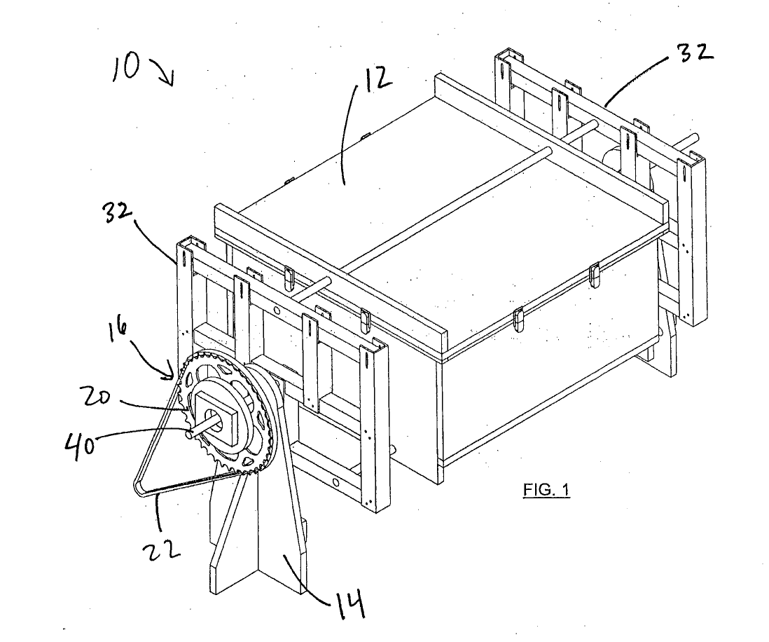

Reference is now made to FIG. 1, in which an example of a device for decellularizing lungs is illustrated generally at 10. Device 10 comprises a vessel 12 that houses a pair of lungs (not shown) during decellularization, a support structure 14, a motorized rotation unit 16, a central axis splitter 32, and a perfusion circuit (not shown). Motorized rotation unit 16 rotates the vessel 12 using a gear 20 and a chain mechanism 22. Chain mechanism 22 may be adopted from similar mechanisms used by bicycles.

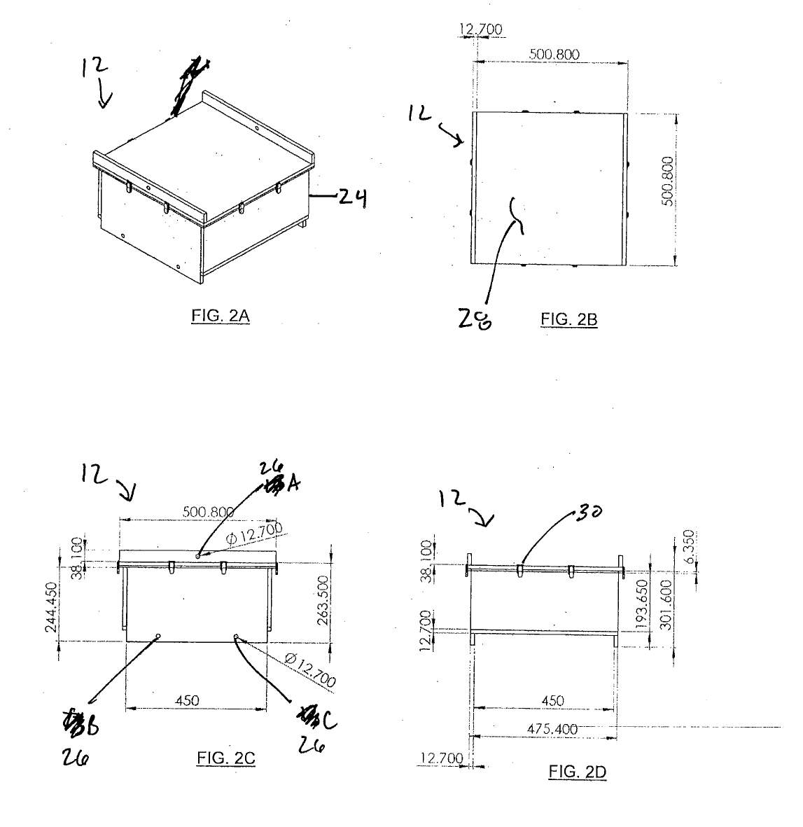

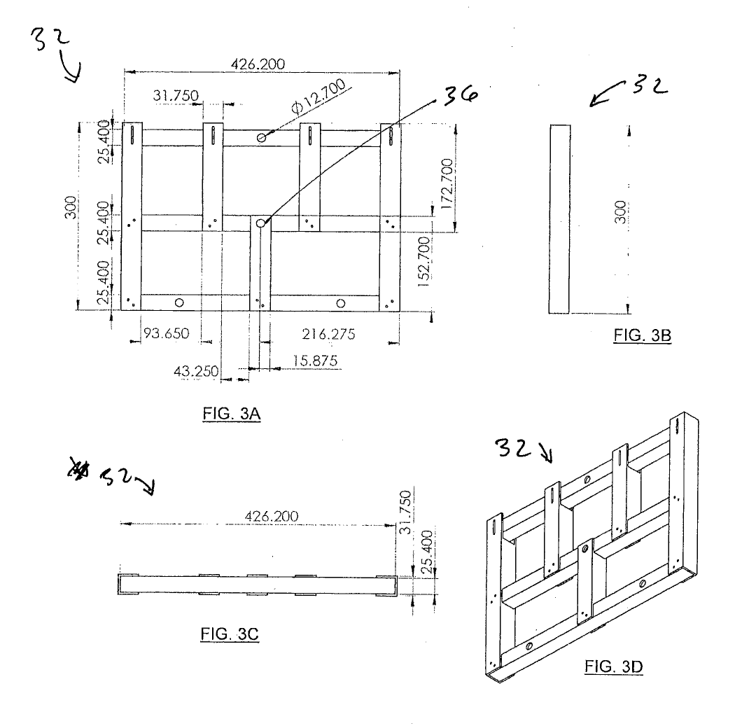

Reference is now made to FIGS. 2A, 2B, 2C, and 2D in which a vessel that holds the lungs during decellularization is illustrated generally at 12. Vessel 12 may be made of a transparent material, such as an acrylic plastic, so that the operator can view the lungs for the duration of the procedure. At each end, the vessel 12 is held at three points 26A, 26B, and 26C for stability by central axis splitter 32, described in FIGS. 3A, 3B, 3C, and 3D. The holding or attachment points can be provided by three rods extending between the central axis splitters 32.

Vessel 12 comprises a rectangular prism 24 that houses the lung (not shown) during decellularization. Vessel 12 rotates under the influence of motorized rotation unit 16 of FIG. 1. The lung is attached to vessel 12 via cannulas (not shown) inserted into the pulmonary artery, pulmonary vein, and trachea of the lung. The cannulas are anchored to rectangular prism 24 as they pass through rectangular prism 24 to connect with the perfusion circuit, located outside of vessel 12. One of the six faces of rectangular prism 24 functions as a lid 28, and may be removed to allow for the insertion and removal of lungs. Lid 28 is held closed using conventional compression buckles 30.

During decellularization, the vessel 12 is filled with a liquid to support the lung. The liquid preferably has a density generally similar to lung tissue, so the lung is uniformly supported, and this may be saline solution or phosphate buffer solution (PBS). Alternatively, plastic or oil based pellets may be used, to fill the space not occupied by the lung, so as to support the lung. The pellets should be compressible under a load of no more than 20 kilograms. A further alternative is a gel that is formed to the shape and size of the lung. Agarose is a gel that may be suitable.

Reference is now made to FIGS. 3A, 3B, 3C, and 3D in which a central axis splitter is illustrated generally at 32. Central axis splitter 32 allows vessel 12 (e.g. of FIGS. 2A, 2B, 2C, and 2D) to rotate about a central axis 36 of the lung, without piercing the structure of the lung with an axle. Central axis splitter 32 allows the lung to rotate about axis 36 that approximately passes through its geometric centroid creating a hydrostatic advantage described below.

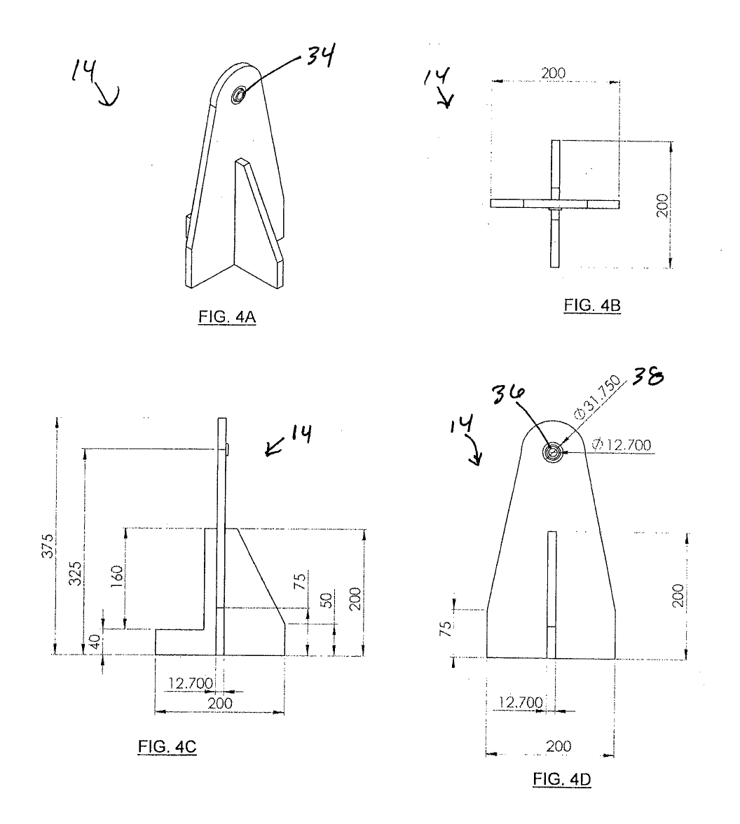

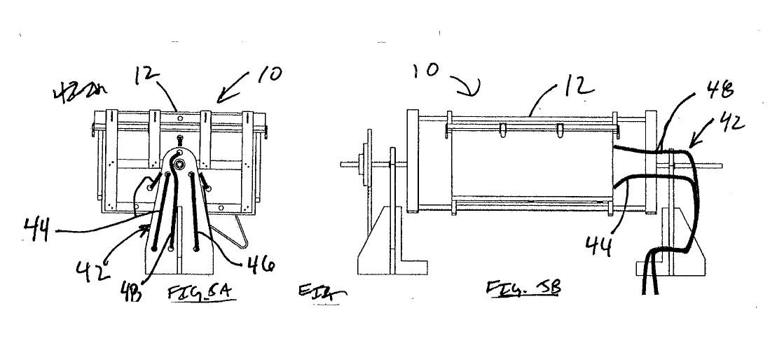

Reference is now made to FIGS. 4A, 4B, 4C, and 4D in which one of two support structures that stand at either end of device 10 is illustrated generally at 14. Support structure 14 elevates vessel 12 (e.g. of FIGS. 2A, 2B, 2C, and 2D) and central axis splitter 32 (e.g. of FIGS. 3A, 3B, 3C, and 3D) above a surface such that they may rotate freely without obstruction. Additionally, support structure 14 transmits the weight of all the components of device 10 to the surface, much like the prier of a bridge. Support structure 14 includes a ball bearing 34 that may be commercially sourced for the axle 36. The larger radius hole 38, with a diameter of 31.75 [mm], reflects the outer diameter of sourced ball bearing 34.

Referring again to FIG. 1, motorized rotation unit 16 is connected to vessel 12 via an axle 40 and gearing mechanism 20. Motorized rotation unit 16 rotates the lung throughout the duration of the decellularization process. The full range of optimal rotation speeds has yet to be determined; a range of 0.5 to 6 rotations per hour (rph) has been found to be effective. Motorized rotation unit 16 may not facilitate a continuous 360 degree rotation. Rather, it may allow vessel 12 to rotate 360 degrees in a clockwise direction, and then prohibit further rotation in the clockwise direction until a 360 rotation in the counterclockwise direction has occurred; other possible limits are 180 degrees and 90 degrees in each direction. This prevents the perfusion tubing from becoming kinked due to excessive twisting, whist still providing a complete rotation for the lung held within the vessel.

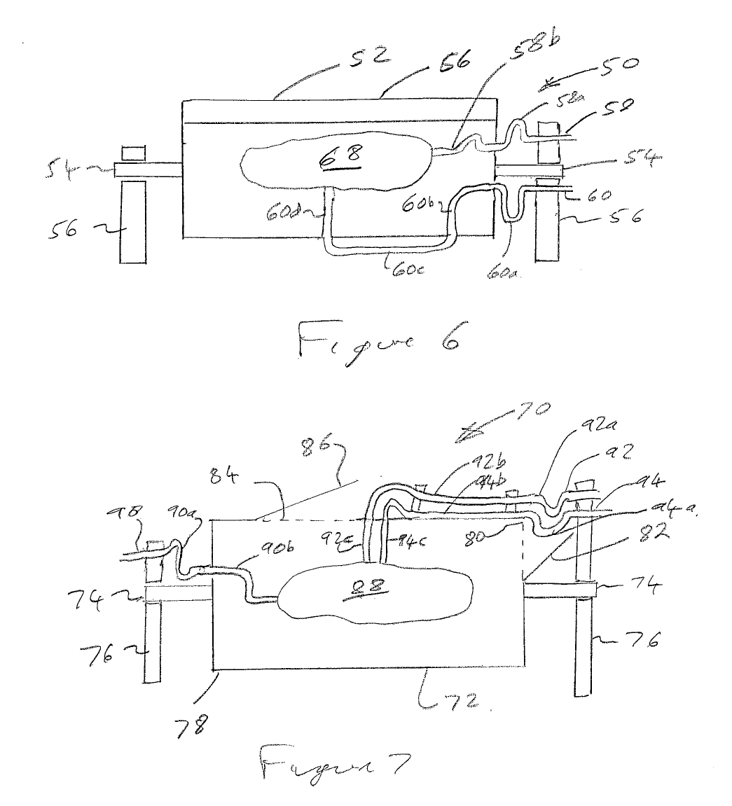

In the embodiments shown, the angular rotation limits may be plus and minus 90 degrees from a central position. FIGS. 1 and 5 show the vessel 12 at one limit position, i.e. the vessel 12 can be rotated through 90 degrees to a position with the lid 28 vertical, and through a further 90 degrees to the other limit position in which the lid 28 would be on the bottom of the vessel 12.

Various timing regimes may be used for the perfusion process or method depending on, for example, the nature and size of the organ subject to perfusion, the process being carried out (which may be other than decellularization), the size of the organ, and the liquid used for perfusion. For example, for a lung a single 15 minute cycle may be sufficient, with the vessel slowly rotated from one limit to the other limit during this period. For other applications, different time periods can be employed, and it may be beneficial to employ multiple cycles of rotating the vessel between the limit positions. It is also anticipated that the vessel and method of the present invention may be used for recellularization.

The perfusion circuit (not shown) is an adaptation of perfusion circuits currently used in the pulmonary case. The perfusion circuit may be adapted from the XVIVO Lung Perfusion system developed by surgeons at the Toronto General Hospital, in Toronto, Canada. The difference between the perfusion circuit used and the XVIVO Lung Perfusion system being the decellularization perfusion circuit does not make use of a leukocyte filter or a gas exchange unit.

Reference is now made to FIGS. 5A and 5B in which the tubing pathway from the outside of device 10 (external) to the inside of vessel 12 that holds the lungs to be perfused is illustrated generally at 42. Tubing pathway 42 comprises venous return tubing 44, pulmonary artery input line 46, and tracheal return/input line 48.

Operational benefits of device 10 will now be discussed in more detail. Device 10 aims to achieve complete perfusion of a human-scale lung whilst using minimal pressure. This results in minimal damage to the extracellular matrix ECM. A more intact the ECM, the better the resulting mass will serve as a scaffold for regenerating organs.

To achieve complete perfusion—that is, to deliver fluid to as many of small capillaries of the vasculature as possible—a minimum vessel pressure required. (Ideally, the fluid is delivered to every capillary, but it has to be accepted that this is usually not possible.) This pressure differs per lung. Initial experiments indicate the fluid pressure required to achieve complete perfusion is approximately 25.5 [mmHg]. Without rotation the lowest section of the lung will be about 7.5 [cm] below the pulmonary artery—the point at which fluid enters the lung. It should be noted this input channel is determined by the physiology of the lung and cannot be changed. The upper most part of the lung will be a further 7.5 [cm] higher than the pulmonary artery. To achieve complete perfusion at the lowest extreme of the lung, the input pressure should be 20 [mmHg]. This implies the pressure at the uppermost extremities of the lung will be a mere 14.5 [mmHg]. This will not achieve complete perfusion. This problem can be overcome by increasing the input pressure such that the highest extremity of the lung receives a fluid pressure of 25.5 [mmHg]. This will, however, expose the lower extremities of the lung to high pressure of approximately 36.5 [mmHg]. This high pressure may inadvertently damage the ECM of the remaining scaffold. Device 10 negates this issue by rotating the lung. This changes which section of the lung is the lower extremity. This exposes each section of the lung to an input pressure combined with the hydrostatic pressure of the decellularization fluid such that the total fluid pressure is 25.5 [mmHg], as desired. No part of the lung is ever exposed to a higher combined fluid pressure, minimizing damage to the ECM as desired.

While the figures illustrate a single device that makes use of the principle described above, it is possible to design a device that makes use of the described principle but that does not resemble the device illustrated in the figures.

A typical dimensions for the device, for use in treatment of lungs can include providing that the access of rotation, i.e. the center access, as located at 325 mm above the support surface. The rectangular prism 24 forming the vessel can have a length and width of approximately 500 mm, and a depth of approximately 200 mm. It will be understood that these dimensions can be varied depending upon the organ to be treated.

While the above description provides examples of one or more processes or apparatuses, it will be appreciated that other processes or apparatuses may be within the scope of the accompanying claims.

With reference to FIG. 6, this shows details of tubing for connecting perfusion circuits to a lung. In FIG. 6, the apparatus is indicated generally at 50, and shows a vessel 52 having a lid 66. The vessel 52 has supporting shafts 54 mounted by bearings in support structures 56.

Tubing for supplying perfusion fluids is mounted in one of the support structures 56. The tubing comprises a tracheal tubing 58 for air, or air equivalent, that is mounted in the support structure 56, and includes a section 58A that is of sufficient length and flexibility to permit rotational movement between the vessel 52 and the support structure 56. The tubing 58 also includes a section 58B within the vessel 52, again of sufficient length and flexibility to accommodate movement of the lung indicated at 68.

A further tubing comprises a pulmonary artery supply tubing 60 for supply of a perfusion fluid, e.g. a surfactant, that is also mounted in the support structure 56. The tubing 60 includes a section 60A of sufficient length and flexibility to permit rotational movement between the vessel 52 and the support structure 56. The tubing 60 extends through the sidewall of the vessel 52 and includes a section 60B within the vessel, that passes through a bottom of the vessel to a section 60C extending outside the vessel and then returning through the vessel to a section 60D extending upwards. This configuration has been adopted since it provides adequate control of the tubing, to avoid kinking and the like, while allowing for the necessary relative motion. The tubing 60D extends upwards and is connected to the lung by a cannula (not shown). Also not shown, there would be a similar venous return tubing for return of perfusion fluid, following generally the same path as the tubing 60.

The configuration shown in FIG. 6 is functional, but may have a disadvantage that the pulmonary arterial flow flow at the onset of flow into the lung or other organ 70 is a flow upwards. This may results in pushing air bubbles into the vasculature. Put another way, any venous return flow from the vasculature may not adequately remove air bubbles, since these will naturally want to move upwards. The arrangement in FIG. 6 was adopted, since the lid 66 is provided at the top of the vessel 52 and is required to be removable.

An alternative configuration is shown in FIG. 7, where the entire apparatus is indicated at 70. The apparatus has a vessel 72 having support shafts 74, again mounted by bearings in support structures 76.

In the embodiment in FIG. 7, the vessel 72 has a main vessel body 78. Rather than providing a distinct and complete lid for the entire top surface of the vessel 72, the vessel 72 has two separate openings 80 and 84, each of which can be closed by a respective closure element 82 and 86. The closure elements 82 and 86 are shown in an open configuration, with their closed configurations indicated by dotted lines in the openings 80 and 84.

The perfusion system for the lung or other organ, indicated at 88, includes a tubing 90, for supply of air or equivalent.

As in FIG. 6, the tubing 90 is mounted in one of the support structure 76, and includes a section 90A, sufficiently long and flexible to enable relative rotation of the vessel 72, and a section 90B within the vessel, for connection to the lung 88.

For the supply of a perfusion fluid, a tubing 92 and a tubing 94 are provided. Each of these tubings 92, 94 includes sections 92A and 94A, also providing for rotation of the vessel 72 relative to the support structure 76. Here, the tubings 92, 94 are shown including sections 92B and 94B mounted on the exterior of the vessel 72. It is possible that the sections 92B, 94B could equally be mounted within the vessel 72.

The tubings 92, 94 also includes sections 92C and 94C, within the vessel 72 and connected by cannulas (not shown) to the lung or other organ 88.

It is anticipated that the advantage of the configuration of FIG. 7 is that the cannulas for the perfusion fluid are then connected to the lung or other organ 88 from above flow at the onset of flow. Consequently, there will be natural tendency for any pockets of air, air bubbles and the like to flow out from the vasculature of the organ. It will be understood that, in the context of providing a flow of perfusion fluid to an organ, small bubbles of air and the like can serve to completely block off flow of the fluid, and it is usually important to ensure that the perfusion circuit is free of any air bubbles and the like.

As the tubing 92 and 94 are now mounted to the vessel 72, the openings 82, 84 are provided for access of the vessel, to enable the lung or other organ 88 to be placed in the vessel, and subsequently surrounded by fluid or other support medium, e.g. pellets and like. Once the lung or other organ 88 is in place and connected by cannulas then the closure elements 82 and 84 can be closed. As for the other embodiments, if the support medium is a fluid, then appropriate seals would be provided for the closure elements 82, 86.
While various embodiments of the invention have been described, the scope of the present invention is determined by the scope of the following claims. In the claims, a number of different features and aspects of the invention are defined and these can be combined together in any practical way having the necessary utility.

We claim:

1. A biomedical device for use in decellularizing, recellularizing or otherwise treating human or animal organs, the device comprising:
(a) a vessel to hold the organ;
(b) a support structure that holds the vessel during rotation; and
(c) a mechanism for rotating the vessel.

2. The biomedical device of claim 1, wherein the device includes a perfusion system for supplying a perfusion fluid, and is adapted to achieve complete perfusion with a low total fluid pressure.

3. The biomedical device of claim 2, wherein the total fluid pressure is developed from a combination of hydrostatic fluid pressure and an input pressure developed by the perfusion system.

4. The biomedical device as claimed in claim 1, including a vessel for holding the organ and a medium for supporting the organ within the vessel.

5. The biomedical device of claim 4, wherein the medium comprises a liquid, and wherein the vessel comprises a vessel body and at least one lid with a seal for sealing the lid to the vessel body.

6. The biomedical device of claim 4, wherein the medium comprises pellets, optionally at least one of plastic or oil based pellets.

7. The biomedical device of claim 1, wherein the support structure provides for limited rotation of the vessel relative to the support structure.

8. The biomedical device of claim 7, wherein the limited rotation provides for rotation of the vessel by 180° in each direction from a neutral position of the vessel.

9. The biomedical device of claim 7, wherein the support structure provides for limited rotation of 90° in each direction from a neutral position of the vessel.

10. The biomedical device of claim 7 including tubing for supply of perfusion fluid connected through the support structure to the vessel.

11. The biomedical device of claim 10, wherein the tubing comprises a supply tubing and a return tubing for connection by cannulas to the vasculature of an organ, for flowing a liquid through the vasculature of the organ.

12. The biomedical device of claim 11, wherein each of the supply tubing and the return tubing is mounted to the support structure, and includes a section between the support structure and the vessel permitting rotation of the vessel relative to the support structure.

13. The biomedical device of claim 12, wherein the vessel comprises a vessel body having a top surface, and wherein the supply tubing and return tubing extend through the top surface downwardly into the vessel for connection to an organ.

14. The biomedical device of claim 13, wherein the vessel includes at least one of an opening in the top surface thereof and an opening in a sidewall of the vessel, for providing access to the interior of the vessel for insertion and removal of an organ and any supporting medium, wherein each opening is provided with a closure element.

15. The biomedical device of claim 1, including cannulas for supplying a perfusion liquid to the organ, wherein, for a lung, the cannulas comprise a pulmonary artery cannula, a pulmonary vein cannula and trachea cannula.

16. A method of supplying perfusion liquid to an organ, the method comprising:
(a) providing for a supply of a perfusion liquid to an organ;
(b) providing for at least limited rotation of the organ; and
(c) while rotating the organ, supplying perfusion liquid to the organ, to cause uniform perfusion of the liquid in the organ.

17. A method as claimed in claim 16, wherein the liquid is supplied at a pressure such that all parts of the organ are subject to the perfusion liquid at a desired minimum pressure.

18. A method as claimed in claim 16, including supporting the organ in a medium in a vessel.

19. A method as claimed in claim 18, wherein the medium comprises one of a liquid and pellets.

20. A method as claimed in claim 16, wherein the organ is rotated at a speed in the range of 0.5 to 6 rotations per hour.

21. A method as claimed in claim 20, wherein the organ is rotated between 180° in either direction from a neutral position in the organ, more preferably between 90° in either direction from a neutral position.

22. A method as claimed in claim 21, including supplying a perfusion liquid through a supply tubing to a vasculature of the organ, and flowing perfusion liquid from the vasculature to the organ through a return tubing.

23. A method as claimed in claim 22, including connecting the supply and return tubing to the organ from above the organ, when the organ is in a neutral position.

24. A method as claimed in claim 23, including supporting the organ in a vessel, and connecting the supply tubing and the return tubing to the vessel.

25. A method as claimed in claim 24, including providing the vessel with at least one access opening in a top surface of the vessel and a sidewall of the vessel, and a closure for each opening.

26. A method as claimed in claim 16, the method being applied to an organ comprising a lung, and the method further comprising providing a tubing for supply of air or an air substitute, the tubing being connected to a trachea of the lung and the method comprising alternately supplying air substitute to the lung and exhausting air or air substitute from the lung, at pressure or flow rate simulating natural respiration.
